(19) United States
(12) Patent Application Publication
(10) Pub. No.: US 2003/0186077 A1
(43) Pub. Date: Oct. 2, 2003

(54) Bis- and tris- (di) benzocarbazole-based materials as hole transport materials for organic light emitting devices

(76) Inventor: Jian P. Chen, San Jose, CA (US)

Correspondence Address: FITZPATRICK CELLA HARPER & SCINTO, 30 ROCKEFELLER PLAZA, NEW YORK, NY, 10112, US

(21) Appl. No.: 10/029,936

(22) Filed: Dec. 31, 2001

(51) Int. Cl. H05B 33/12; C07D 209/56; C 07D 4 9/00

(52) U.S. Cl. 428/690; 428/917; 313/504; 313/506; 548/416; 548/418; 548/420; 548/517

(57) ABSTRACT

Compounds according to Formula I

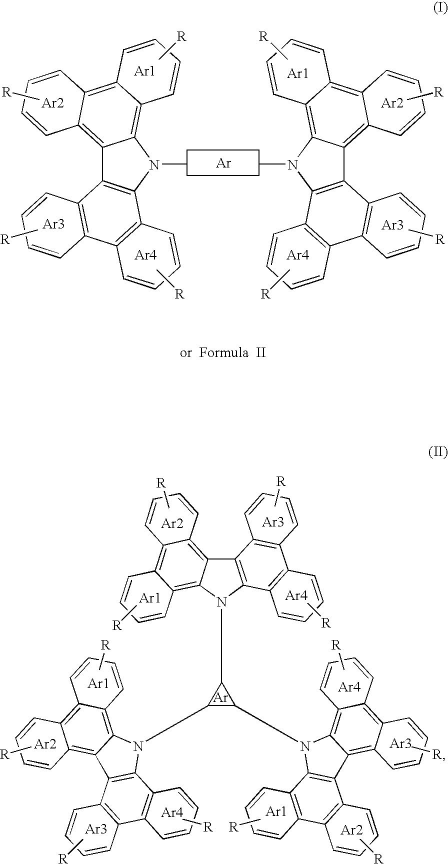

wherein one or two of Ar1-4 are present, R is selected from the group consisting of H, CH₃, OCH₃ or halogen, and Ar is an aryl bridge. Such compounds are suitable for use in organic light-emitting devices. These compounds exhibit both hole transport and emissive properties and have a high glass transition temperature and thermal stability.

### BACKGROUND OF THE INVENTION

1. Field of the Invention

The present invention relates to bis- and tris-(di)benzocarbazole-based materials suitable for use in organic light emitting devices (OLEDs). The invention further relates to methods of making such bis- and tris-(di)benzocarbazole-based materials and to OLEDs employing such bis- and tris-(di)benzocarbazole-based materials in an emissive or hole transport layer.

2. Description of the Related Art

In general, OLEDs are useful as full-color, flat-panel displays and are advantageous in that they exhibit low voltage driving, high brightness and RGB color emission. Typically, an OLED consists of a single or multiple emission layers of organic thin film(s) sandwiched between a transparent anode and a vacuum-evaporated metal cathode. Upon a forward bias, holes are injected from anodes and electrons from cathodes, respectively. Transport of injected charge carriers, and recombination of holes and electrons in the emission layer, generate photons which emit light.

One factor influencing high performance in an OLED is charge balance. Generally, layered devices consisting of charge-transport and emission layers can more readily achieve charge balance than single-layer devices. This is because a suitable combination of charge-transporting and emitting materials in layered devices reduces the energy barrier for the injection of charge carriers from the electrodes. Additionally, the charge-transport layer acts as a blocking layer against the injection of either holes or electrons from the adjoining layer and their subsequent escape from the device.

It has therefore been considered to insert a hole transport layer between an anode and an emission layer to assist hole injection and to block electrons. Hole transport materials have good hole conduction characteristics and have a small energy barrier for hole injection from the anode. Triarylamines are well known organic hole transport materials for use in OLEDs. In particular, N,N′-diphenyl-N,N′-di-m-tolyl-1,1′-biphenyl-4,4′-diamine (TPD) and N,N′-di-1-naphthyl-N,N′-diphenyl-1,1′-biphenyl-4,4′-diamine (NPD), and their analogs, are widely used.

Although triarylamines exhibit excellent hole transport properties, they generally produce amorphous films with poor morphological stability due to their low glass transition temperature (Tg). For example, TPD has a Tg of 65° C. OLEDs comprising hole transport layers made from materials having such a low Tg exhibit poor thermal stability. The low Tg hole transport materials dewet from other interface layers at device operation conditions and can even crystallize at room temperature absent operation of the device. Such OLED degradation detracts from both OLED performance and OLED lifetime.

### SUMMARY OF THE INVENTION

It is an object of the present invention to provide materials suitable for use in the emission or hole transport layer of multi-layer OLEDs and exhibiting excellent thermal stability and a high glass transition temperature.

Preferably, embodiments of the invention include compounds which are bis- and tris-dibenzocarbazoles and bis- and tris-benzocarbazoles.

In accordance with this invention, compounds according to Formula 1

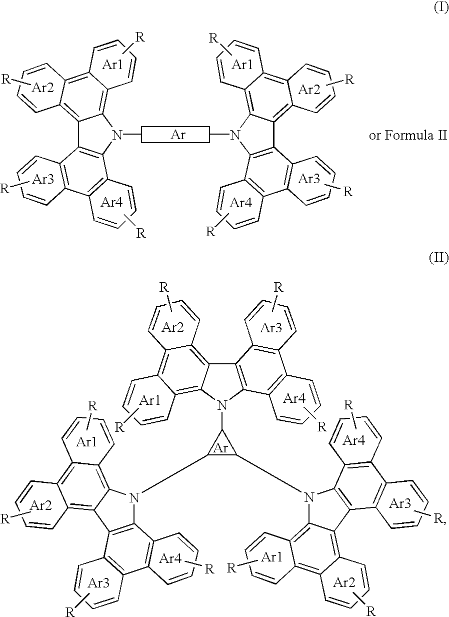

wherein one or two of Ar1-4 are present, R is selected from the group consisting of H, CH₃, OCH₃ or halogen, and Ar is an aryl bridge, are useful.

In further aspects, the present invention is an organic light-emitting device comprising a transparent anode, a cathode, and an emission layer containing a compound of formula (I) or (II) or an organic light emitting device comprising a transparent anode, a cathode, an emission layer, and a hole transport layer containing a compound of formula (I) or (II).

In a related aspect, the invention is a method of making a compound of formula (I) comprising the step of reacting one equivalent of a diiodo- or dibromo-aromatic compound and two equivalents of (di)benzocarbazole in the presence of a metal catalyst. The present invention is also directed to a method of making a compound of formula (II) comprising the step of reacting one equivalent of a triiodo- or tribromo-aromatic compound and three equivalents of (di)benzocarbazole in the presence of a metal catalyst.

This brief summary has been provided so that the nature of the invention may be understood quickly. A more complete understanding of the invention can be obtained by reference to the following detailed description of the preferred embodiments thereof in connection with the attached drawings.

### BRIEF DESCRIPTION OF THE DRAWING

FIG. 1 is a cross-sectional view of an organic light emitting device according to the invention.

FIG. 2 contains graphs showing the characteristics of OLED's made using compounds according to the present invention.

### DETAILED DESCRIPTION OF THE PREFERRED EMBODIMENTS

In one aspect, the invention is a compound having the structure

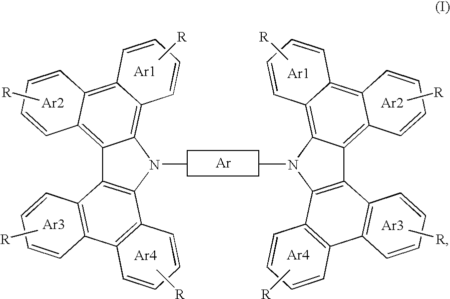

wherein R is selected from the group consisting of H, CH₃, OCH₃ or halogen, and Ar is an aryl bridge. Preferably, exactly one or exactly two of the Ar1 through Ar4 are present.

In certain preferred embodiments, Ar is selected from

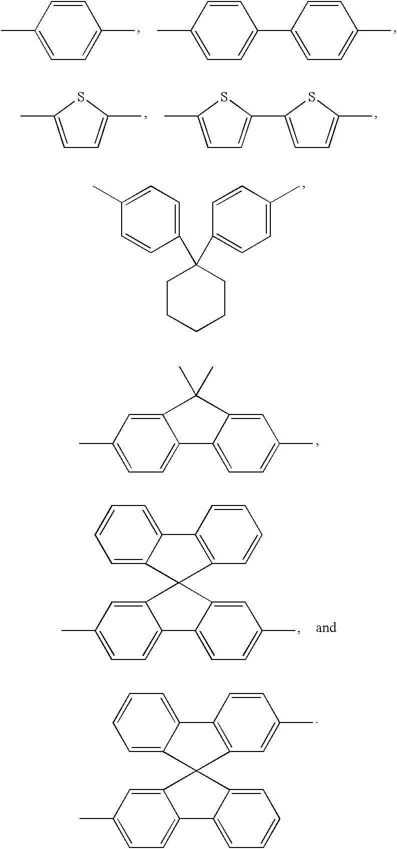

Certain preferred embodiments of the present invention are directed to compounds which are bis-dibenzocarbazoles and bis-benzocarbazoles. Among the bis-dibenzocarbazoles are 1,4-bis-7H-dibenzo[c,g]carbazole-benzene, 4,4′-bis-7H-dibenzo[c,g]carbazole-biphenyl, 2,7-bis-7H-dibenzo[c,g]carbazole-9,9-dimethyl-fluorene, 4,4′-bis-13H-dibenzo[a,i]carbazole-biphenyl, 4,4′-bis-13H-dibenzo[a,g]carbazole-biphenyl, 4,4′-bis-2,12-dimethoxy-7H-dibenzo[c,g]carbazole-biphenyl, 2,7-bis-2,12-dimethoxy-7H-dibenzo[c,g]carbazole-9,9-dimethyl-fluorene, 1,4-bis-2,12-dimethoxy-13H-dibenzo[c,g]carbazole-benzene, 1,4-bis-2,12-dimethoxy-7H-dibenzo[c,g]carbazole-benzene, and 1,4-bis-13H-dibenzo[a,i]carbazole-benzene.

Among the bis-benzocarbazoles are 4,4′-bis-7H-benzo[c]carbazole-biphenyl, 1,4-bis-7H-benzo[c]carbazole-benzene, 4,4′-bis-11H-benzo[a]carbazole-biphenyl, 1,4-bis-11H-benzo[a]carbazole-benzene, 1,3-bis-7H-benzo[c]carbazole-benzene, 1,3-bis-11H-benzo[a]carbazole-benzene, and 4,4′-bis-3-phenyl-11H-benzo[a]carbazole-biphenyl.

In another aspect, the present invention is directed to a compound having the structure

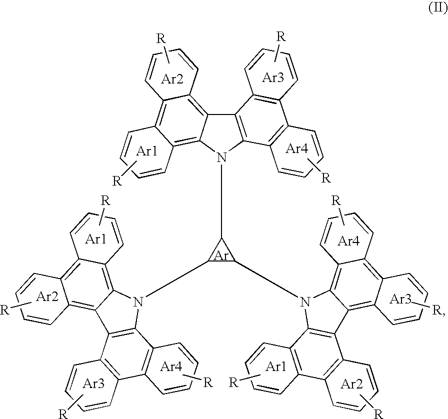

wherein R is selected from the group consisting of H, CH₃, OCH₃ or halogen, and Ar is an aryl bridge. Preferably, exactly one or exactly two of the Ar1 through Ar4 are present.

In certain preferred embodiments, Ar is selected from

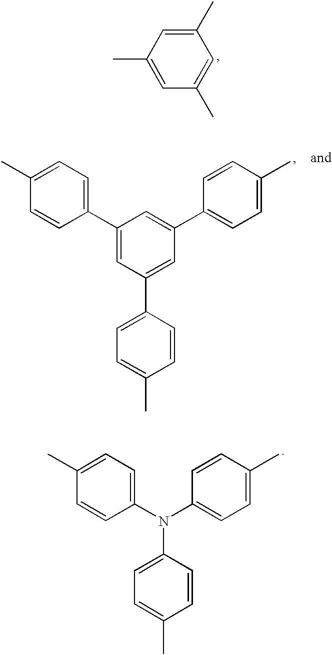

Certain preferred embodiments of the present invention are directed to compounds which are tris-dibenzocarbazoles and tris-benzocarbazoles. Among the tris-dibenzo carbazoles are 4,4′,4″-tris(N-dibenzo[a,g]carbazolyl)triphenylamine. Among the tris-benzocarbazoles are 4,4′,4″-tris(N-benzo[c]carbazolyl)triphenylamine and 4,4′,4″-tris(N-benzo[a]carbazolyl)triphenylamine.

The above-described compounds exhibit excellent thermal stability and have high glass transition temperatures. As a result, OLEDs incorporating such bis- and tris-benzo- and dibenzocarbazole-based materials as emissive and/or hole transport materials exhibit improved lifetimes.

Synthesis of Starting Materials

The starting materials used to synthesize the compounds according to Formula I or Formula II of the present invention can be made using known procedures or according to the methods outlined below.

7H-Dibenzo[c,g]carbazole, 13H-dibenzo[a,i]carbazole and 11H-benzo[a]carbazole, 7H-benzo[c]carbazole are synthesized according to the procedures described in Katritzky A. R. and Wang Z., Journal of Heterocyclic Chemistry, 25, 671, 1988, which is incorporated by reference herein. 2,7-diiodo-fluorene is synthesized according to the procedures described in Bansal R. C. et al, OPPI Briefs, V.19, No.2-3, 258-261 (1987). The Bansal et al. paper is also incorporated by reference herein.

Using the compounds described above, additional starting materials can be made as follows:

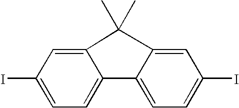

9,9-Dimethyl-2,7-diiodo-fluorene: Mixtures of 2,7-diiodo-fluorene (1 equiv.), iodomethane (2.2 equiv., 2M in t-butylmethylether) and NaOt-Bu (2.2 equiv.), were stirred in DMF at 10° C. under N₂ for 2 h. The solution was poured into water and the precipitate was formed. The product was filtrated, washed with water and air-dried. It was purified through a silica-gel column with hexanes. yield was 85%. Mp. 195° C.

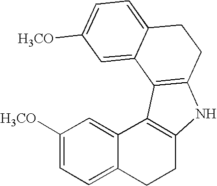

2,12-Dimethoxy-5,6,8,9-tetrahydro-7H-dibenzo[c,g]carbazole: 7-methoxy-2-tetralone (9.7 g, 55 mmol, Fw 176), H₂NNH₂.H₂O (1.35 mL, 27.5 mmol, d 1.027, Fw 50), HAc (0.5 mL), ethanol (10 mL) were placed into a flask. The mixture was refluxed under N₂ overnight. After cooling, the solids were filtrated, washed with ethanol, water, and ethanol, then dried under vacuum. Yield: 8.0 g (87%). Mp. 206° C.

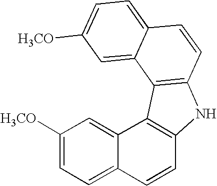

2,12-Dimethoxy-7H-dibenzo[c,g]carbazole: 2,12 Dimethoxy-5,6,8,9-tetrahydro-7H-dibenzo[c,g]carbazole (3 g) and 5% palladium charcoal (1 g) were heated at 250-260° C. for 30 min. The mixture was then extracted with benzene. After removal of solvent, the product solidified upon addition of hexanes. Yield: 2.5 g (84%). Mp. 133° C.

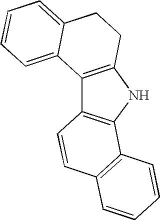

1,2-Dihydro-13H-Dibenzo[a,g]carbazole: This compound was synthesized using 1-naphthylhydrazine and 2-tetralone in the presence of HCl in refluxing acetic acid. Mp. 218° C.

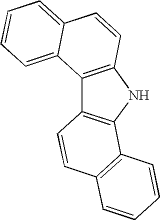

13H-Dibenzo[a,g]carbazole: 1,2-Dihydro-13H-Dibenzo[a,g]carbazole was dehydrogenated with 5% palladium charcoal to provide 13H-Dibenzo[a,g]carbazole. Mp. 238° C.

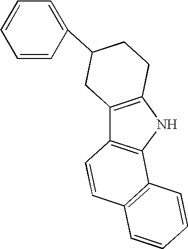

3-Phenyl-1,2,4-trihydro-11H-benzo[a]carbazole: This compound was synthesized using 1-naphthylhydrazine and 4-phenylcyclohexanone in the presence of HCl in refluxing acetic acid. Mp. 198° C.

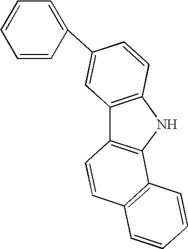

3-Phenyl-11H-benzo[a] carbazole: 3-Phenyl- 1,2,4-trihydro-11H-benzo[a]carbazole was dehydrogenated with 5% palladium charcoal to afford 3-Phenyl 11H-benzo[a]carbazole. Mp. 254° C.

Applying the general scheme of the present invention and the starting materials just described, compounds of formula (I) are made by reacting one equivalent of a diiodo- or dibromo-aromatic compound and two equivalents of (di)benzocarbazole in the presence of a metal catalyst. Further, compounds of formula (II) are made by reacting one equivalent of a triiodo- or tribromo-aromatic compound and three equivalents of (di)benzocarbazole in the presence of a metal catalyst.

Suitable metal catalysts are known to these of ordinary skill and include copper powder and dipalladium-tris (dibenzylindeneacetone).

The reactions are conducted under a nitrogen atmosphere with refluxing over a time period ranging from overnight to two days.

Further details and aspects of the method of making the compounds in accordance with this invention are found in the specific examples provided below.

These examples of the preferred embodiments of the bis- and tris- (di) benzocarbazole materials and the OLEDs incorporating them are for illustration purposes, and are not to be considered limiting of the invention, which is defined by the claims.

Bis and Tris-(Di)Benzocarbazole Syntheses

### EXAMPLE 1

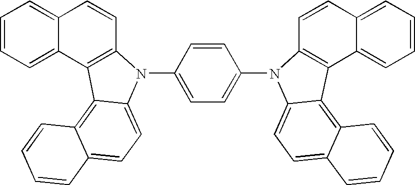

1,4,-bis-7H-Dibenzo[c,g]carbazole-benzene: 7H-Dibenzo[c,g]carbazole (Fw 267, 0.534 g, 2 mmol), 1,4-diiodobenzene (Fw 330, 0.33 g, 1 mmol), copper powder (Fw 63.5, 0.127 g, 2 mmol), K₂CO₃ (Fw 138, 0.552 g, 4 mmol), 18-Crown-6 (Fw 264, 40 mg, 0.15 mmol) were placed in a dry round-bottle flask connected with a condenser. The system was evacuated and purged with N₂ for at least 2 cycles. Under N₂, 1,2-dichlorobenzene (6 mL, b.p. 180° C.) was added and the mixture was refluxed for 2 days. The hot solution was filtrated through a bed of silica to remove solids. The filtrate was drop-added into methanol and the precipitate was filtrated, washed with methanol, and dried at 70° C. under vacuum. Yield: 0.50 g (82%); IR (neat), 1510, 1394, 792 cm⁻¹; TG, Td=409° C., DSC, Tm=352° C.

### EXAMPLE 2

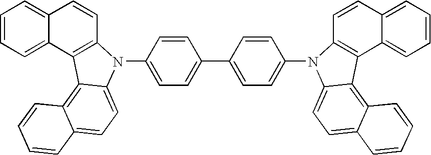

4,4′-bis-7H-Dibenzolc,glcarbazole-biphenyl: This compound was synthesized using the same conditions as Example 1, by using two equivalents 7H-dibenzo[c,g]carbazole and one equivalent 4,4′-diiodobiphenyl in place of 1,4-diiodobenzene. Yield, 87%; IR (neat), 1502, 796, 742 cm⁻¹; TGA, Td>350° C.

### EXAMPLE 3

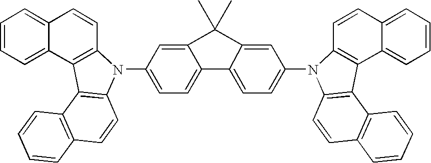

2,7-bis-7H-Dibenzo[c,g]carbazole-9,9-biphenyl-fluorene: This compound was synthesized using the same conditions as Example 1, by using two equivalents 7H-dibenzo[c,g]carbazole and one equivalent 2,7-diiodo-9,9-dimethylfluorene in place of 1,4-diiodobenzene. Yield, 87%; TGA, Td>400° C.

### EXAMPLE 4

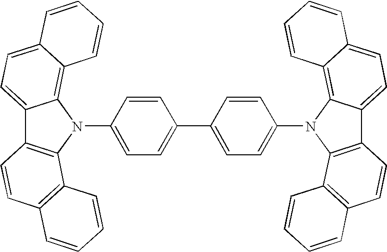

4,4′-bis-13H-Dibenzo[a,i]carbazole-biphenyl: This compound was synthesized using the same conditions as Example 1, by using two equivalents 13H-dibenzo[a,i]carbazole and one equivalent 4,4′-diiodobiphenyl. Yield, 87%; IR (neat), 1495, 1378, 800, 738 cm⁻¹; TGA, Td>400° C.

### EXAMPLE 5

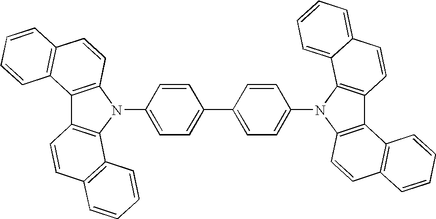

4,4′-bis-13H-Dibenzo[a,g]carbazole-biphenyl: A round-flask was charged with 4,4′-diiodobiphenyl (0.406 g, 1 mmol), 13H-dibenzo[a,g]carbazole (0.534 g, 2 mmol), NaOt-Bu (0.23 g, 2.4 mmol), tris(dibenzylideneacetone)-dipalladium (0) (Pd₂dba₃, 36 mg, 4% mol), 2-(di-t-butylphosphino)-biphenyl (36 mg, 12% mol) and o-xylene (15 mL). The mixture was refluxed under N₂ overnight. After cooling down, the mixture was poured into methanol. The precipitate was filtrated, washed with water and methanol, and air-dried. The solids were then dissolved in hot 1,2-chlorobenzene and filtrated off the insoluble parts. The filtrate was then drop-added into methanol. The precipitate was filtrated and dried under vacuum. Yield, 85%. TGA, Td=360° C.

### EXAMPLE 6

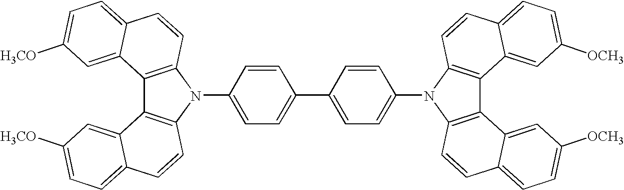

4,4′-bis-2,12-Dimethoxy-7H-Dibenzo[c,g]carbazole-biphenyl: This compound is synthesized using the same conditions as example 1, by using two equivalents 2,12-dimethoxy-7H-dibenzo[c,g]carbazole and one equivalent 4,4′-diiodobiphenyl. TGA, Td>350° C.

### EXAMPLE 7

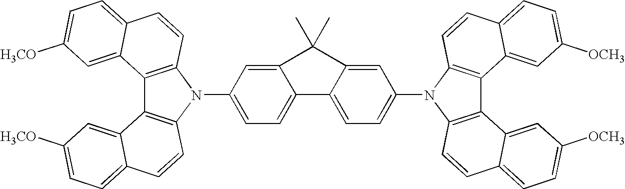

2,7-bis-2,12-Dimethoxy-7H-Dibenzo[c,g]carbazole-9,9-dimethyl-fluorene: This compound was synthesized using the same conditions as Example 1, by using two equivalents 2,12-dimethoxy-7H-dibenzo[c,g]carbazole and one equivalent 9,9-dimethyl-2,7-diiodo-fluorene. TGA, Td=402° C.

### EXAMPLE 8

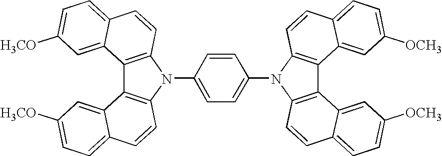

1,4,-bis-2,12-Dimethoxy-13H-Dibenzo[c,g]carbazole-benzene: This compound was synthesized using the same conditions as Example 1, by using two equivalents 2,12-dimethoxy-7H-dibenzo[c,g]carbazole and one equivalent 1,4-diiodobenzene. TGA, Td=404° C., DSC, Tm=358° C.

### EXAMPLE 9

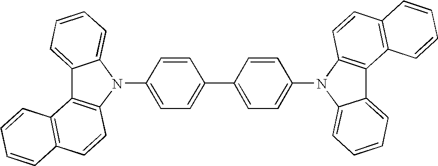

4,4′-bis-7H-benzo[c]carbazole-biphenyl: This compound was synthesized using the same conditions as Example 1, by using two equivalents 7H-benzo[c]carbazole and one equivalent 4,4′-diiodobiphenyl. TGA, Td=410° C.

### EXAMPLE 10

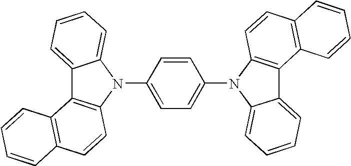

1,4,-bis-7H-benzo[c]carbazole-benzene: This compound was synthesized using the same conditions as Example 1, by using two equivalents 7H-benzo[c]carbazole and one equivalent 1,4-dliodobenzene. TGA, Td=408° C.

### EXAMPLE 11

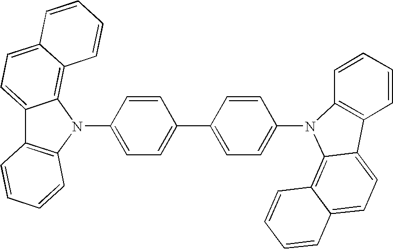

4,4′-bis-11H-benzo[a]carbazole-biphenyl: This compound was synthesized using the same conditions as Example 1, by using two equivalents 11h-benzo[a]carbazole and one equivalent 4,4′-diiodobiphenyl. TGA, Td=366° C.

### EXAMPLE 12

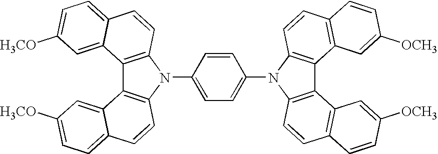

1,4-bis-2,12-Dimethoxy-7H-Dibenzo[c,g]carbazole-benzene: This compound was synthesized using the same conditions as Example 1, by using two equivalents 2,12-dimethoxy-7H-dibenzo[c,g]carbazole and one equivalent 1,4-diiodobenzene. TGA, Td=404° C.; DSC, Tm=358° C.

### EXAMPLE 13

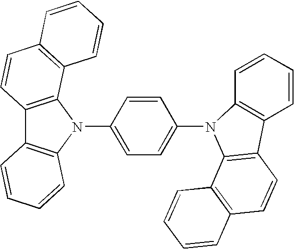

1,4,-bis-11H-benzo[a]carbazole-benzene: This compound was synthesized using the same conditions as Example 1, by using two equivalents 11H-benzo[a]carbazole and one equivalent 1,4-diiodobenzene. TGA, Td=395° C.; DSC, Tm=380° C.

### EXAMPLE 14

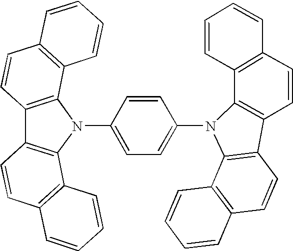

1,4,-bis-13H-dibenzo[a,i]carbazole-benzene: This compound was synthesized using the same conditions as Example 1, by using two equivalents 13H-dibenzo[a,i]carbazole and one equivalent 1,4-diiodobenzene. TGA, Td=340° C.

### EXAMPLE 15

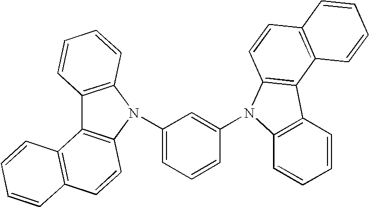

1,3,-bis-7H-benzo[c]carbazole-benzene: This compound was synthesized using the same conditions as Example 1, by using two equivalents 7H-benzo[c]carbazole and one equivalent 1,3-diiodobenzene. TGA, Td=399° C.; DSC, Tg=113° C.

### EXAMPLE 16

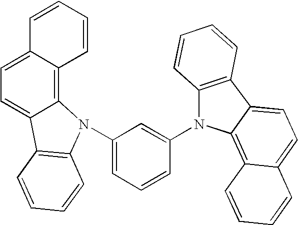

1,3,-bis-11H-benzo[c]carbazole-benzene: This compound was synthesized using the same conditions as Example 1, by using two equivalents 11H-benzo[a]carbazole and one equivalent 1,3-diiodobenzene. TGA, Td=330° C.; DSC, Tm=248° C.

### EXAMPLE 17

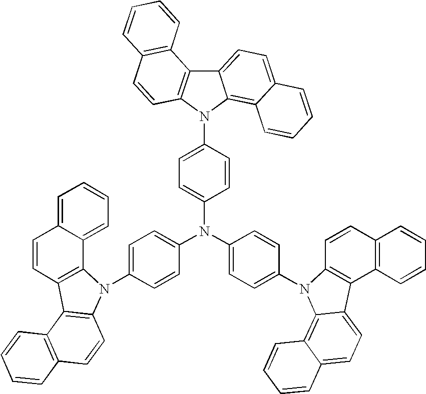

4,4′,4″-tris(N-dibenzo[a,g]carbazolyl)triphenylamine: This compound was synthesized using the same conditions as Example 5, by using three equivalents 13H-dibenzo[a,g]carbazole and one equivalent tris(4-bromophenyl)amine. TGA, Td=585° C.; DSC, Tg=212° C.

### EXAMPLE 18

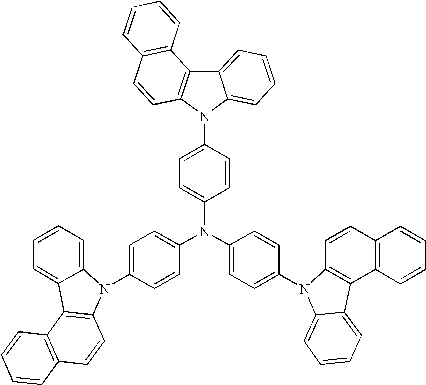

4,4′,4″-tris(N-benzo[c]carbazolyl)triphenylamine: 7H-benzo[c]carbazole (Fw 217, 0.651 g, 3 mmol), tris(4-bromophenyl)amine (Fw 482, 0.482 g, 1 mmol), copper powder (Fw 63.5, 50 mg), K₂CO₃ (Fw 138, 1.0 g), were placed in a dry round-bottle flask connected with a condenser. The system was evacuated and pevacuated and purged with N₂for at least 2 cycles. Under N₂, dry nitrobenzene (10 mL, b.p. 210° C.) was added and the mixture was refluxed for one day. The hot solution was filtrated and the filtrate was drop-added into methanol. The precipitate was filtrated, washed with methanol, and water. It was re-dissolved into toluene and flash-column purified. After concentrating the toluene solution, the product precipitated from methanol. After filtration, product was dried at 70° C. under vacuum. TGA, Td=475° C., DSC, Tg=158° C.

### EXAMPLE 19

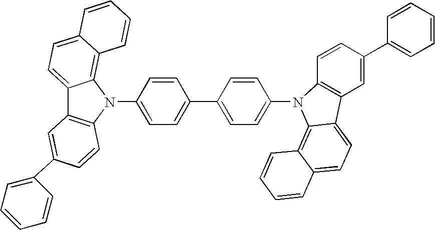

4,4′-bis-3-Phenyl-11H-benzo[a]carbazole-biphenyl: This compound was synthesized using the same conditions as Example 1, by using two equivalents 3-phenyl-11H-benzo[a]carbazole and one equivalent 4,4′-diiodobiphenyl. TGA, Td=366° C.

### EXAMPLE 20

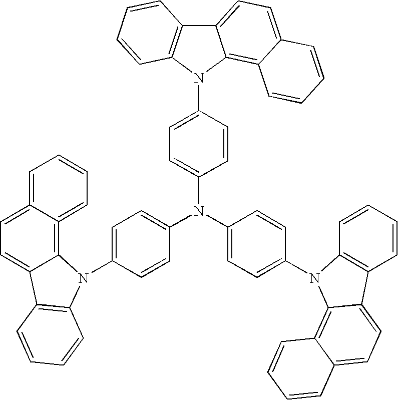

4,4′,4″-tris(N-benzo[a]carbazolyl)triphenylamine: This compound was synthesized using the same conditions as Example 20, by using three equivalents 11H-benzo[a]carbazole and one equivalent tris(4-bromophenyl)amine. TGA, Td=366° C.

OLEDS Device Fabrications and Characterizations

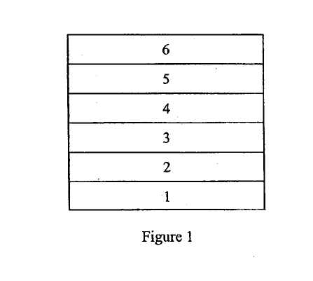

FIG. 1 schematically depicts a device according to the invention, including transparent substrate 1, anode 2 adjacent the substrate, hole transport layer 3 based on the bis- or tris- (di)benzcarbazole compounds of the present invention adjacent the anode, an optional emissive layer 4, an electron transport layer 5 adjacent the emissive layer, and cathode 6. Each of these layers may itself comprise multiple layers of material having similar composition or function.

Devices according to the invention find use in display applications such as television screens, computer screens and image bar components for digital copiers and printers.

Suitable materials for substrate 1 include glass, quartz and the like, and polymers (including, without limitation, polyesters, polycarbonates, polyacrylates, polymethacrylates, and polysulfones). The thickness of the substrate is not critical and can range, for example, from about 25 to over 1,000 microns, depending on the structural demands of the device.

The anode adjacent the substrate can be comprised of a metal, an alloy, an electroconducting compound, or mixtures thereof, especially with a work function equal to, or greater than about 4 electron volts. Specific examples of anodes include positive charge injecting electrodes such as indium tin oxide (ITO), tin oxide, zinc oxide, gold, platinum, electrically conductive carbon, and conjugated polymers such as polyaniline, polypyrrole, and the like. ITO is preferred. The thickness of the anode can range anywhere from about 10 nanometers to 1 micron.

The hole injecting layer may be comprised of a layer comprising bis- or tri-(di) benzcarbazole hole transport components in accordance with the present invention. In certain embodiments, an emissive/hole transporting layer can be used instead of a hole transport layer. Thus, the hole transport layer 3 is optional.

As an optional electron-transport layer any known electron transport materials can be used. AlQ type materials, such as tris-(8-hydroxyquinoline)aluminum and derivatives thereof are particularly preferred.

In embodiments of the present invention, the total thickness of the combined emissive, hole transport and electron transport layers is less than about 1 micron, and preferably is from about 400 angstroms to about 4000 angstroms, sufficient to maintain a current density that permits efficient light emission under a relatively low voltage applied across the electrodes. Suitable thickness of the layers can range preferably from about 50 to about 2,000 angstroms, and preferably from about 400 to 1,000 angstroms.

The cathode 6 can be comprised of any metal, including high or low work function metals. Aluminum, lithium, magnesium and calcium are particularly preferred.

Referring again to the reference numerals of FIG. 1, an OLED according to the invention may be made by first cleaning a glass substrate 1 having an ITO anode 2 patterned thereon in isopropyl alcohol for 5 minutes, followed by rinsing in deionized water for 5 minutes and in isopropyl alcohol again for an additional 5 minutes. The cleaned ITO substrate is placed in a substrate holder of a vacuum deposition chamber and the pressure is reduced to about 2×10⁻⁴ Pa, A layer of bis or tris (di) benzocarbazoles in accordance with this invention is then deposited. An electron transport layer 5 such as AlQ3 is then deposited, followed by a LF/Al cathode 6, for example. A driving voltage is applied and a pure color emission is observed.

More specific embodiments which are illustrative of the present invention are as follows:

### EXAMPLE 21

Fabrication of an OLED by Using Compound 1 as a Hole Transporter

An ITO (indium tin oxide) patterned (25 mm×75 mm×1 mm) glass substrate was cleaned with isopropyl alcohol for 5 minutes by using ultrasonic waves, with pure water for 5 minutes, and finally with isopropyl alcohol for 5 minutes. The cleaned ITO substrate was held in a substrate holder for vacuum deposition. After the pressure in the apparatus was reduced to 2×10⁻⁴ Pa, compound 1 (1,4-bis-7H-dibenzo[c,g]carbazole-benzene) was heated to deposit on the ITO substrate at a deposition rate of 3 nm/sec to a thickness of 50 nm. Then tris(8-hydroxyquinoline)aluminum (AlQ3) was then deposited onto the hole transport layer substrate at a deposition rate of 3 nm/sec. The AlQ3 layer had a thickness of 60 nm. Finally, LiF (0.8 nm) was deposited, followed by deposition of Al (200 nm) to form cathodes. A driving voltage was applied to the obtained device by connecting the ITO electrode to a positive electrode(+) and the cathode of LiF/Al to a negative electrode(−) to test emission of light, and a uniform light of green color was obtained. The following device data were obtained: the density of electric current: 600 mA/cm² at 15 V, the luminance: 6600 cd/m² at 15 V, and the maximum external quantum efficiency: 0.8%, the maximum external power efficiency 0.45 lm/W or 2.0 cd/A.

### EXAMPLE 22

Fabrication of an OLED by Using Compound 2 as a Hole Transporter

An OLED device was constructed using the same procedure as Example 21 using 4,4′-bis-7H-Dibenzo[c,g]carbazole-biphenyl (compound 2) as the hole transporter. The following device data were obtained: the density of electric current: 800 mA/cm² at 15 V, the luminance: 16600 cd/m² at 15 V, and the maximum external quantum efficiency: 1.5%, the maximum external power efficiency 1.0 lm/W or 2.7 cd/A.

### EXAMPLE 23

Fabrication of an OLED by Using Compound 3 as a Hole Transporter

An OLED device was constructed using the same procedure as Example 21 using 2,7bis-7H-Dibenzo[c,g]carbazole-9,9-dimethyl fluorene (compound 3) as the hole transporter. The following device data were obtained: the density of electric current: 350 mA/cm² at 15 V, the luminance: 9000 cd/m² at 15 V, and the maximum external quantum efficiency: 2.0%, the maximum external power efficiency 0.55 Im/W or 5.2 cd/A.

### EXAMPLE 24

Fabrication of an OLED by Using Compound 8 as a Hole Transporter

An OLED device was constructed using the same procedure as Example 21 using 1,4-bis-2,12-Dimethoxy-13H-Dibenzo[c,g]carbazole-benzene (compound 8) as the hole transporter. The following device data were obtained: the density of electric current: 450 mA/cm² at 15 V, the luminance: 5500 cd/m² at 15 V, and the maximum external quantum efficiency: 1.0%, the maximum external power efficiency 0. 12 lm/W or 2.2 cd/A.

### EXAMPLE 25

Fabrication of an OLED by Using Compound 14 as a Hole Transporter

An OLED device was constructed using the same procedure as Example 21 using 1,4-bis-13H-dibenzo[a,i]carbazole-benzene (compound 14) as the hole transporter. The following device data were obtained: the density of electric current: 450 mA/cm² at 15 V, the luminance: 1600 cd/M² at 15 V, and the maximum external quantum efficiency: 0.20%, the maximum external power efficiency 0.15 lm/W or 0.6 cd/A.

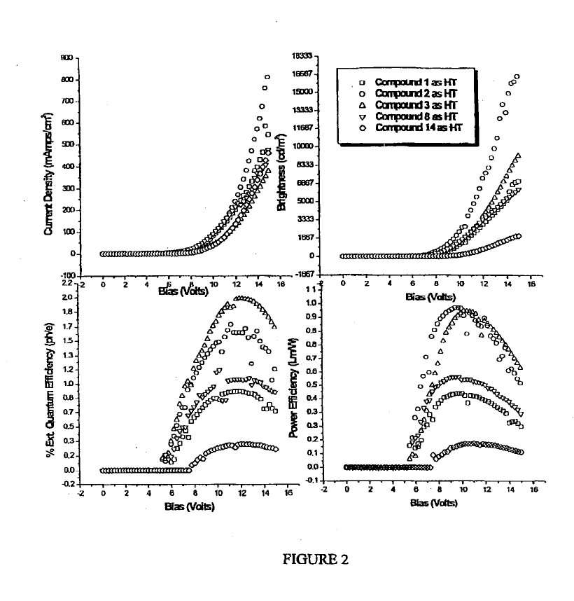

FIG. 2 graphically shows the data obtained by using the compounds of Examples 1, 2, 3, 8 and 14 in the OLEDs of Examples 21-25.

These examples are provided solely for illustration and are not intended to limit the scope of the invention.

What is claimed is:

1. A compound having the structure of Formula I 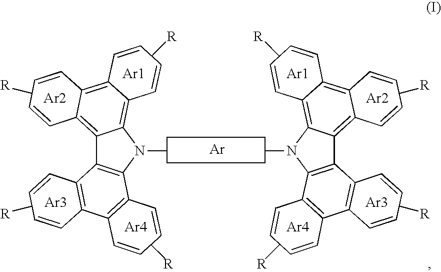
wherein one or two of Ar1-4 are present, R is selected from the group consisting of H, CH₃, OCH₃ or halogen, and Ar is an aryl bridge.

2. The compound of claim 1, wherein Ar is selected from the group consisting of

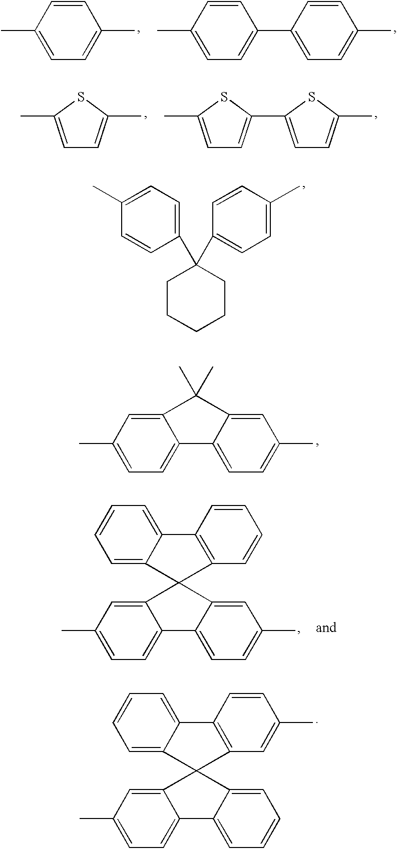

3. The compound of claim 1, wherein the compound is a bis-dibenzocarbazole.

4. The compound of claim 3, wherein the compound is selected from the group consisting of 1,4,-bis-7H-dibenzo[c,g]carbazole-benzene, 4,4′-bis-7H-dibenzo[c,g]carbazole-biphenyl, 2,7-bis-7H-dibenzo[c,g]carbazole-9,9-dimethyl-fluorene, 4,4′-bis-13H-dibenzo[a,i]carbazole-biphenyl, 4,4′-bis-13H-dibenzo[a,g]carbazole-biphenyl, 4,4′-bis-2,12-dimethoxy-7H-dibenzo[c,g]carbazole-biphenyl, 2,7-bis-2,12-dimethoxy-7H-dibenzo[c,g]carbazole-9,9-dimethyl-fluorene, 1,4-bis-2,12-dimethoxy- 13H-dibenzo[c,g]carbazole-benzene, 1,4bis-2,12-dimethoxy-7H-dibenzo[c,g]carbazole-benzene, and 1,4-bis-13H-dibenzo[a,i]carbazole-benzene.

5. The compound of claim 1, wherein the compound is a bis-benzocarbazole.

6. The compound of claim 5, wherein the compound is selected from the group consisting of 4,4′-bis-7H-benzo[c]carbazole-biphenyl, 1,4-bis-7H-benzo[c]carbazole-benzene, 4,4′-bis-11H-benzo[a]carbazole-biphenyl, 1,4-bis-11H-benzo[a]carbazole-benzene, 1,3-bis-7H-benzo[c]carbazole-benzene, 1,3-bis-11H-benzo[a]carbazole-benzene, and 4,4′-bis-3-phenyl- 11H-benzo[a]carbazole -biphenyl.

7. A compound having the structure of Formula II 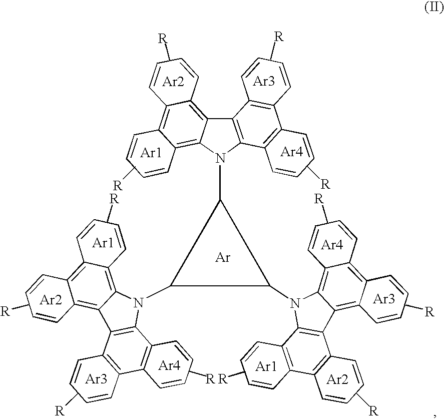
wherein one or two of Ar1-4 are present, R is selected from the group consisting of H, CH₃, OCH₃ or halogen, and Ar is an aryl bridge.

8. The compound of claim 7, wherein Ar is selected from the group consisting

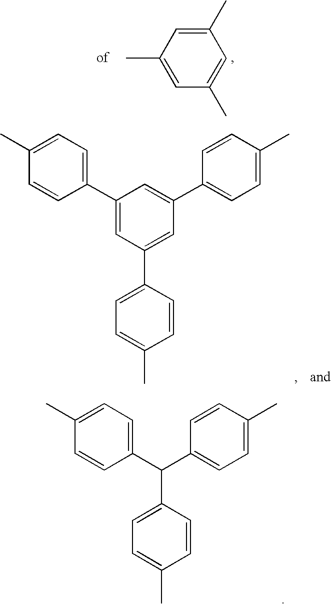

9. The compound of claim 7, wherein the compound is a tris-dibenzocarbazole.

10. The compound of claim 9, wherein the compound is 4,4′,4″-tris(N-dibenzo[a,g]carbazolyl)triphenylamine.

11. The compound of claim 7, wherein the compound is a tris-benzocarbazole.

12. The compound of claim 11, wherein the compound is selected from the group consisting of 4,4′,4″-tris(N-benzo[c]carbazolyl)triphenlyamine and 4,4′,4″-tris(N-benzo[a]carbazolyl)triphenylamine.

13. A method of making a compound according to claim 3 comprising the step of reacting one equivalent of a diiodo- or dibromo-aromatic compound and two equivalents of dibenzocarbazole in the presence of a metal catalyst.

14. A method of making a compound according to claim 5 comprising the step of reacting one equivalent of a diiodo- or dibromo-aromatic compound and two equivalents of benzocarbazole in the presence of a metal catalyst.

15. A method of making a compound according to claim 9 comprising the step of reacting one equivalent of a triiodo- or tribromo-aromatic compound and three equivalents of dibenzocarbazole in the presence of a metal catalyst.

16. A method of making a compound according to claim 11 comprising the step of reacting one equivalent of a triiodo- or tribromo-aromatic compound and three equivalents of benzocarbazole in the presence of a metal catalyst.

17. An organic light emitting device comprising:
(a) a transparent anode;
(b) a cathode; and
(c) an emission layer containing a compound according to any one of claims 1-12.

18. An organic light emitting device comprising:
(a) a transparent anode;
(b) a cathode;
(c) an emission layer; and
(d) a hole transport layer containing a compound according to any one of claims 1-12.
